(19) United States
(12) Patent Application Publication
(10) Pub. No.: US 2014/0212688 A1
(43) Pub. Date: Jul. 31, 2014

(54) HIGH GRADE TITANIUM ALLOY SHEET AND METHOD OF MAKING SAME

(71) Applicant: AMETEK, Inc., Berwyn, CT (US)

(72) Inventors: Ryan Smith, Cheshire, CT (US); Mark Robinson, West Lawn, PA (US); Muktesh Paliwal, Brookfield, CT (US); David Englehart, Shillington, PA (US); Kerry B. Daley, Meriden, CT (US)

(73) Assignee: AMETEK, Inc., Berwyn, PA (US)

(21) Appl. No.: 14/168,501

(22) Filed: Jan. 30, 2014

Related U.S. Application Data
(60) Provisional application No. 61/759,111, filed on Jan. 31, 2013.

(51) Int. Cl. A61L 27/06; B21B 1/22

(52) U.S. Cl. CPC A61L 27/06; B21B 1/22
USPC 428/687; 428/544; 72/203

(57) ABSTRACT

A sheet of titanium alloy that is less than 0.015″ thick has adequate plasticity for subsequent forming into at least a part of a medical device that is MR-Conditional. A method for making the titanium alloy sheet includes cold rolling a titanium alloy to form a sheet having an average thickness less than or equal to 0.015″, cutting the sheet to length, and vacuum annealing the sheet in a final step, wherein the sheet following vacuum annealing has adequate plasticity for subsequent forming into at least a part of a medical device that is MR-Conditional. Neither grinding nor chemical etching is used to reduce the thickness of the titanium alloy when processing the sheet.

### CROSS-REFERENCE TO RELATED APPLICATION

This application claims priority benefit of U.S. Provisional Patent Application Ser. No. 61/759,111, filed Jan. 31, 2013, the entire disclosure of which is expressly incorporated by reference herein.

### FIELD OF THE INVENTION

The invention relates to titanium alloy sheets and methods of making titanium alloy sheets that may be used in the manufacture of medical devices, including surgical implants.

### BACKGROUND OF THE INVENTION

Medical devices which are surgically implanted into the body are highly regulated to ensure the safety of the humans or animals in which the devices are inserted. The materials used to manufacture the devices must be biologically compatible to avoid negative reactions that may occur when inside the patient's body. Therefore, a limited number of materials may be approved for use in such applications. Certain titanium alloys have been found to be acceptable for use in surgically implanted devices.

From the approved group of materials, an alloy must be selected that also has the physical properties which would enable the metal alloy sheet to withstand exposure to stresses during processing. An alloy which does not have adequate plasticity may not withstand the manufacturing processes used to form the components of the device. The manufacturing processes may include rolling, stamping, bending, stretching, spinning, and deep drawing. Inadequate plasticity will result in fracturing or cracking of the sheet before it has been formed into the final desired configuration.

A material commonly used in the fabrication of medical devices, such as pacemakers, is Commercially Pure (CP) Titanium, also referred to as Titanium Grades 1 through 4. The popularity of CP Ti is primarily due to its high plasticity making it an easier material to form into desired shapes. However, there is a shift in the medical industry away from the use of CP Ti to higher grade alloys, such as Grade 5, also known as Ti6Al4V, Ti-6Al-4V or Ti 6-4, and Grade 23, also known as Ti6Al4V ELL These higher grade alloys offer added benefits. For example, medical devices that are made from Grade 5 or Grade 23 titanium alloys are safe for use in a Magnetic Resonance Imaging (MRI) machine, do not limit the distance from which telemetric measurements may be taken. Moreover, for devices that include a battery, such as a pacemaker, the alloys do not heat when placed in a magnetic field typically used to recharge a battery externally by induction. The higher grade alloys however have the drawback of being less ductile than CP Ti.

Many medical devices include parts that are made from titanium alloy sheet that is less than 0.5 mm (0.020″) thick. Common methods employed to produce such thin sheets of Grade 5 or Grade 23 titanium alloys include the use of grinding media or chemical etching. Both methods are wasteful in that they achieve a sheet having a desired thickness by stripping away material. The use of grinding media especially may create unwanted stresses in the sheet, negatively affecting the plasticity of the sheet. Chemical etching requires the use of hazardous chemicals, such as hydrofluoric acid, and may charge the titanium alloy with hydrogen, causing it to become brittle. Both methods are limited in the ability to produce a sheet with a relatively smooth surface having a uniform thickness.

Therefore, there is a need for an improved method of making light gauge Grade 5 or Grade 23 titanium alloy sheets.

### SUMMARY OF THE INVENTION

It is an aspect of the invention to provide a non-ground, non-chemically milled sheet of Grade 5 or Grade 23 titanium alloy that is less than 0.020″ thick, preferably less than 0.015″.

One embodiment of the invention is a metal sheet comprising a titanium alloy, the sheet having an average thickness less than or equal to 0.015″ and sufficient plasticity for stamping, drawing, or deep drawing the sheet into at least a part of a medical device that is MR-Conditional.

As used throughout the specification and claims, “MR-Conditional” means a device or implant that may contain magnetic, electrically conductive, or RF-reactive components that is safe for operations in proximity to an MRI machine.

Another embodiment of the invention is a method of making light gauge metal sheet comprising cold rolling a titanium alloy to form a titanium alloy sheet having an average thickness less than or equal to 0.015″, cutting the titanium alloy sheet to length, and vacuum annealing the titanium alloy sheet in a final step, wherein the titanium alloy sheet following vacuum annealing has sufficient plasticity for stamping, drawing, or deep drawing the sheet into at least a part of a medical device that is MR-Conditional.

Yet another embodiment of the invention is a method of making light gauge metal sheet consisting essentially of cold rolling a titanium alloy to form a titanium alloy sheet having an average thickness less than or equal to 0.015″ and vacuum annealing the titanium alloy sheet in a final step, wherein the titanium alloy following vacuum annealing has sufficient plasticity for stamping, drawing, or deep drawing the sheet into at least a part of a medical device that is MR-Conditional.

### BRIEF DESCRIPTION OF THE FIGURES

In order that the invention may be more fully understood, the following figures are provided by way of illustration, in which:

FIG. 1a is a photomicrographic image of the microstructure of hot pack rolled Grade 23 titanium alloy which has not been subjected to a process of the present invention; and

FIG. 1b is a photomicrographic image of the microstructure of the Grade 23 titanium alloy of FIG. 1a following processing according to an embodiment of the present invention.

### DETAILED DESCRIPTION

Grade 5 and Grade 23 titanium alloy sheet is currently manufactured by hot pack rolling of hot rolled slabs. Titanium mill processing includes the steps of melting and forging the metal into hot rolled sheet bar which is subsequently hot-pack cross-rolled to a thickness of approximately 0.020″. The sheet is then annealed and undergoes surface grinding and pickling, but these steps do not greatly reduce the thickness of the sheet. Therefore, the final thickness of sheet made by current processes is limited to approximately 0.020″.

As previously mentioned, to satisfy needs for strip in thicknesses below 0.020″, current practice is to either mechanically grind or chemically mill the sheet produced by hot-pack rolling to reduce the thickness to the desired value. The primary advantage of the present invention is that it allows for the manufacture of thinner Grade 5 or Grade 23 sheet with suitable plasticity than is currently available from titanium alloy suppliers and eliminates the need to buy oversized sheet and mechanically grind or chemically mill the sheet to reduce its thickness and therefore improves material yield by up to 75% for material processed to 0.005″.

To manufacture Grade 5 or Grade 23 strip having a thickness below that which is currently commercially available, a process has been discovered within the scope of the invention that is able to reduce 0.020″ thick hot-pack rolled sheet to less than 0.015″, more preferably to a thickness between 0.002″ and 0.015″, and most preferably to a thickness between 0.0039″ and 0.012″. The resulting strip has plasticity characteristics which allow it to be formed into small, complex parts typical of medical devices or surgical implants. Preferably, “sufficient plasticity for stamping, drawing, or deep drawing the sheet into at least a part of a medical device” is the extent to which a solid material can be plastically deformed without fracture. The processes of stamping, drawing, and deep drawing, and the extent to which they place stresses on a sheet when making a medical device, are well-known in the art. Thus, the process of the present invention enables the fabrication of metal sheet which has sufficient plasticity to endure any currently known process of stamping, drawing, and deep drawing (including the most strenuous such processes) used to make a medical device or a part thereof, and preferably at least one of the following medical devices: Implantable cardioverter-defibrillators, implantable neurostimulators, implantable pulse generators, bone plates, cardiac valve prostheses, pacemakers, artificial hearts, cochlear implants, dental implants, and dental implant prosthesis components. More preferably, “sufficient plasticity for stamping, drawing, or deep drawing the sheet into at least a part of a medical device” is measured and expressed as tensile elongation according to ASTM F 136 and is at least 10%. Most preferably, “sufficient plasticity for stamping, drawing, or deep drawing the sheet into at least a part of a medical device” is measured by the Erichsen Ball Punch Deformation Test according to ASTM E643 and for a 0.010″ thick sheet is at least an average of 5 mm at failure, more preferably, at least an average of 5.5 mm, and most preferably at least an average 6.0 mm.

The process includes cold rolling the hot pack rolled sheet to reduce the thickness to the finish thickness. To make subsequent forming of parts possible, the cold rolled sheet is then vacuum annealed to recrystallize the microstructure and restore plasticity to the sheet. If the desired thickness is thinner than approximately 50% of the starting hot pack rolled sheet thickness, the process may include more than one cycle of cold rolling the sheet to an intermediate thickness and vacuum annealing until the final desired thickness is obtained.

The process according to the invention preferably does not produce Grade 5 or Grade 23 sheet in the form of a coil. It is preferred to maintain the sheet in a flat orientation thereby enabling either longitudinal and transverse rolling, or both. Also, such an orientation allows the sheet to be immediately and easily cut to dimensions specified by a medical device or surgical implant manufacturer, i.e. Cut-to-Length (CTL) process steps may be associated with a process according to the present invention which are known to those of skill in the art.

Transverse rolling is optional in the process according to the present invention. Transverse rolling is performed by first passing a sheet through a set of mill rolls in the longitudinal direction, then turning the sheet 90 degrees after reducing its thickness and feeding the sheet again through the mill rolls in the transverse direction.

Hot-pack rolled sheet is currently only available in sheets of approximately 100″ in its longest dimension (i.e. length). This limitation arises because the hot-pack rolling procedure involves cross rolling, which limits the finished length' of the hot-pack rolled sheet to that of the width of the hot rolling mill. However, the process according to the present invention does not require cross rolling and may produce sheets up to 10 times longer than sheet that is currently available, as would be the case where a 0.020″ thick hot-pack rolled sheet is cold rolled to a finish thickness of 0.002″.

It is preferred that the process of the present invention include a cutting step prior to the final annealing step. No grinding or chemical etching of the sheet is required, and no oil or lubrication is needed during cold rolling.

It is preferred that the cold rolling step included in a process according to the present invention comprises one or more passes through a pair of rollers and the average thickness of the titanium alloy sheet prior to the cold rolling step is reduced by at least 35% upon completion of the cold rolling step, more preferably the average thickness is reduced to between 35% and 70%, and most preferably between 40% and 60%. It is preferred that the average thickness of the sheet per pass is reduced by 1% to 10%.

It is preferred that cold rolling comprises feeding the titanium alloy through a pair of rolls and is conducted between a temperature of 0° C. and 100° C., more preferably between 5° C. and 80° C., and most preferably between 10° C. and 65° C. The distance between the pair of rolls is adjusted after each pass. The rolls may be made of any standard material known to those of skill in the art, such as tool steel or carbide. As the sheet approaches its final thickness, it is preferred that the final rolls of the mill used in the cold rolling process of the present invention utilize a pair of tool steel rolls. Such rolls are known in the art.

As mentioned above, the process according to the invention may comprise one cold-rolling step comprising several single passes followed by vacuum annealing or may comprise a plurality of cold-rolling steps followed by vacuum annealing after each step. Vacuum annealing is the final step prior to producing a Grade 5 or Grade 23 titanium alloy sheet having the target thickness. Thus, one embodiment of the present invention includes a method of making light gauge metal sheet comprising cold rolling a titanium alloy to form a titanium alloy sheet having an average thickness less than or equal to 0.015″, cutting the titanium alloy sheet, wherein the titanium alloy sheet is cut to length, and vacuum annealing the titanium alloy sheet in a final step, wherein the cutting step is performed prior to the vacuum annealing step and the titanium alloy sheet following vacuum annealing has sufficient plasticity for stamping, drawing, or deep drawing the sheet into at least a part of a medical device that is MR-Conditional. The methodology for determining the average thickness of a metal sheet is well-known in the art and includes all or substantially all of a sample of the sheet, but excludes any region of a tapered edge;

In an alternative embodiment of the present invention, the process consists essentially of cold rolling a titanium alloy to form a titanium alloy sheet having an average thickness less than or equal to 0.015″, wherein the titanium alloy is suitable for the manufacture of a medical device that is MR-Conditional; and vacuum annealing the titanium alloy sheet in a final step. This embodiment would exclude certain process steps, such as the process step of interleaving the titanium alloy with a material, such as iron aluminide, during heat treatment.

According to one embodiment of the present invention, it is preferred that the vacuum annealing occur at a pressure less than or equal to 10⁻³ torr, more preferably less than or equal to 10⁻⁴ torr, and most preferably less than or equal to 10⁻⁵ torr. The annealing may also occur under an inert atmosphere. It is preferred that the vacuum annealing be conducted below the beta transus temperature of the titanium alloy sheet, most preferably at 750° C. for two hours.

As previously mentioned, vacuum annealing the titanium alloy sheet according to the present invention recrystallizes the microstructure and restores plasticity to the sheet.

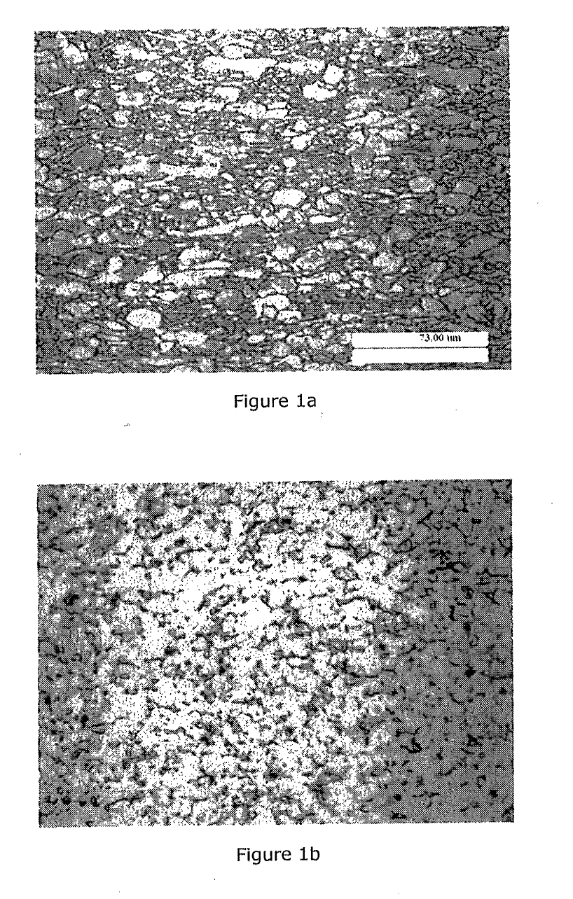

According to ASTM F 136, the microstructure for titanium alloy used in medical devices and surgical implants is required to exhibit no continuous alpha network at prior beta grain boundaries. Comparison of FIGS. 1a and 1b demonstrates that a Grade 23 titanium alloy sheet made according to one embodiment of the present invention meets this standard. FIG. 1b is a photomicrographic image of Grade 23 titanium alloy which has been cold rolled and vacuum annealed according to an embodiment of the present invention. The light etching phase is the alpha phase, and the darker etching phase is the beta phase. From the image, it is apparent that the beta phase is well distributed in the alpha, in contrast to the coarser and more globular microstructure shown in the photomicrographic image of the same sample prior to cold rolling and vacuum annealing as shown in FIG. 1a.

Also within the scope of the invention is metal sheet made from a process according to the invention. Another embodiment of the invention includes metal sheet comprising a titanium alloy, the sheet having an average thickness less than or equal to 0.015″ and plasticity for subsequent forming into at least a part of a medical device or implant that is MR-Conditional. Some of the devices or implants that may be made from the metal sheet include implantable cardioverter-defibrillators, implantable neurostimulators, implantable pulse generators, bone plates, cardiac valve prostheses, pacemakers, artificial hearts, cochlear implants, dental implants, and dental implant prosthesis components.

Another advantage of the metal sheet within the scope of the invention is that the metal sheet exhibits significantly less variation in thickness from location to location in the sheet as compared to mechanically ground or chemically milled sheet. This tighter thickness tolerance is of great benefit to stamping and forming operations which are used to convert the flat titanium alloy sheet into parts such as neurostimulator enclosures. It is preferred that the thickness along the length of a sheet of titanium alloy according to one embodiment of the invention does not deviate more than 10% of the average thickness, more preferably 8.5%, and most preferably 7.5%.

The Grade 5 and Grade 23 titanium alloy sheet according to the present invention exhibits greater plasticity as compared to hot-pack rolled titanium alloy sheet of similar grade. The metal sheet according to the present invention has the benefit of metallurgical properties making it adequate for forming of the sheet into parts of a medical device or implant. ASTM F 136 sets the standards for determining whether titanium alloys may be used to manufacture medical devices and surgical implants. According to ASTM F 136, the metal sheet according to the present invention exhibits a tensile elongation greater than or equal to 10%, a yield strength of at least 115 ksi, and an ultimate tensile strength of at least 125 ksi; thus, embodiments of the metal sheet of the present invention meet the minimum standards needed for the manufacture of parts of a medical device or surgical implant. The methodology for measuring and determining a value for each of “tensile elongation,” “yield strength,” and “ultimate tensile strength” is specifically set out in ASTM F 136.

The metal sheet according to the present invention contains fewer stresses which further enhances the plasticity of the sheet for drawing operations. The lack of stresses is attributed to the process used to make the metal sheet which does not require the use of grinding media to achieve the thicknesses below 0.015″. Grinding media can cause surface cracking and stresses thereby degrading the plasticity of the metal sheet. As a result, the surface of the metal sheet according to the present invention is free of embedded grinding media, is free of surface residual stresses or surface cracks, and exhibits a smoother surface topography compared to titanium alloy strip made by mechanically grinding hot pack rolled sheets. This smoother surface eliminates the directional scratches or grinding marks typical of mechanically ground sheet which can initiate cracking or failure during subsequent forming of the sheet into parts. It is preferred that the metal sheet according to the present invention have a surface roughness of 2 Ra to 50 Ra, more preferably a surface roughness of 2.5 Ra to 25 Ra, and most preferably a surface roughness of 3 Ra to 15 Ra. The methodology and instruments for measuring and determining a value for “surface roughness” is well-known in the art. For example, the surface roughness may be measured using the MarSurf M300 surface measuring device manufactured by Mahr GmbH of Gottingen, Germany.

The enhanced plasticity of the metal sheet according to the present invention is also attributed to the vacuum annealing step used to produce the metal sheet. The titanium alloy metal sheet of the present invention exhibits a more uniform microstructure than hot pack rolled sheet, as illustrated in FIGS. 1a and 1b, as a result of the vacuum annealing step. A more uniform microstructure leads to improved plasticity. The vacuum annealing step also results in a metal sheet according to the present invention which comprises significantly lower hydrogen content compared to hot pack rolled or chemically milled titanium alloy sheet. Hydrogen is known to embrittle titanium alloys, so a reduced hydrogen level is beneficial to the plasticity of the sheet. It is preferred that the titanium alloy sheet of the present invention have a maximum hydrogen content of 0.015% wt., more preferably a maximum hydrogen content of 0.010% wt., and most preferably, a maximum hydrogen content of 0.004% wt. The methodology for measuring and determining a value for “hydrogen content” is well-known in the art.

While preferred embodiments of the invention have been shown and described herein, it will be understood that such embodiments are provided by way of example only. Numerous variations, changes, and substitutions will occur to those skilled in the art without departing from the spirit of the invention. Accordingly, it is intended that the appended claims cover all such variations as fall within the spirit and scope of the invention.

What is claimed:

1. A metal sheet comprising a titanium alloy, the sheet having an average thickness less than or equal to 0.015″ and sufficient plasticity for stamping, drawing, or deep drawing the sheet into at least a part of a medical device or implant that is MR-Conditional.

2. The metal sheet of claim 1, wherein the titanium alloy is Grade 23 titanium alloy.

3. The metal sheet of claim 1 having a tensile elongation greater than or equal to 10%.

4. The metal sheet of claim 1, wherein a sample of the metal sheet having a thickness of 0.010″ exhibits an average value of at least 5.0 mm at failure when subjected to an Erichsen Ball Punch Deformation Test.

5. The metal sheet of claim 1 having a yield strength of at least 115 ksi.

6. The metal sheet of claim 1 having an ultimate tensile strength of at least 125 ksi.

7. The metal sheet of claim 1 having a surface roughness of 2 Ra to 50 Ra.

8. The metal sheet of claim 1 having a minimum thickness of 0.002″.

9. The metal sheet of claim 1 that is free of surface residual stresses or surface cracks.

10. The metal sheet of claim 1, wherein all surfaces of the sheet are free of embedded grinding media.

11. The metal sheet of claim 1, wherein the sheet has a maximum thickness less than or equal to 107.5% of the average thickness.

12. The metal sheet of claim 1, wherein the sheet has a minimum thickness less than or equal to 92.5% of the average thickness.

13. The metal sheet of claim 1, wherein the sheet has a maximum hydrogen content of 0.015% wt.

14. The metal sheet of claim 1, wherein the sheet has a maximum hydrogen content of 0.004% wt.

15. The metal sheet of claim 1, wherein the medical device or implant is selected from the group consisting of implantable cardioverter-defibrillators, implantable neurostimulators, implantable pulse generators, bone plates, cardiac valve prostheses, pacemakers, artificial hearts, cochlear implants, dental implants, and dental implant prosthesis components.

16. A medical device or implant made from a metal sheet according to claim 1.

17. A method of making light gauge metal sheet comprising;
cold rolling a titanium alloy to form a titanium alloy sheet having an average thickness less than or equal to 0.015″;
cutting the titanium alloy sheet, wherein the titanium alloy sheet is cut to length; and
vacuum annealing the titanium alloy sheet in a final step,
wherein the cutting step is performed prior to the vacuum annealing step and the titanium alloy sheet following vacuum annealing has sufficient plasticity for stamping, drawing, or deep drawing the sheet into at least a part of a medical device or implant that is MR-Conditional.

18. The method of claim 17, wherein the cold rolling step comprises a plurality of passes through mill rolls and the average thickness of the titanium alloy sheet prior to the cold rolling step is reduced by at least 35% upon completion of the cold rolling step.

19. The method of claim 17, wherein the titanium alloy is cold rolled in a transverse direction and in a longitudinal direction.

20. The method of claim 17, wherein the titanium alloy is Grade 23 titanium alloy.

21. The method of claim 17, wherein the titanium alloy sheet has a maximum thickness less than or equal to 107.5% of the average thickness.

22. The method of claim 17, wherein the titanium alloy sheet has a minimum thickness less than or equal to 92.5% of the average thickness.

23. The method of claim 17, wherein the cold rolling step is conducted between a temperature of 0° C. and 100° C.

24. The method of claim 17, wherein the vacuum annealing step is conducted below the beta transus temperature of the titanium alloy sheet.

25. The method of claim 17, wherein following the vacuum annealing step, the titanium alloy sheet has a hydrogen content less than 0.004% wt.

26. A metal sheet made according to the method of claim 17.

27. A medical device made from the metal sheet of claim 26.

28. A method of making light gauge metal sheet consisting essentially of:
cold rolling a titanium alloy to form a titanium alloy sheet having an average thickness less than or equal to 0.015″; and
vacuum annealing the titanium alloy sheet in a final step,
wherein the titanium alloy sheet following the vacuum annealing step has sufficient plasticity for stamping, drawing, or deep drawing the sheet into at least a part of a medical device that is MR-Conditional.
